(19) United States
(12) Patent Application Publication
(10) Pub. No.: US 2023/0255928 A1
(43) Pub. Date: Aug. 17, 2023

(54) METHODS OF INCREASING MICROVASCULAR BLOOD FLOW

(71) Applicant: Abbott Laboratories, Abbott Park, IL (US)

(72) Inventors: Suzette PEREIRA, Westerville, OH (US); Ricardo RUEDA CABRERA, Granada (ES); Vikkie MUSTAD, Galena, OH (US)

(21) Appl. No.: 17/996,793

(22) PCT Filed: Apr. 23, 2021
(86) PCT No.: PCT/US2021/028756

§ 371 Date: Oct. 21, 2022

Related U.S. Application Data
(60) Provisional application No. 63/014,727, filed on Apr. 24, 2020.

(51) Int. Cl. A61K 31/353; A61P 7/00; A23L 33/105; A23L 33/00; A23L 33/19; A23L 33/185; A23L 33/175; A23L 33/125; A23L 33/115

(52) U.S. Cl. CPC A61K 31/353; A61P 7/00; A23L 33/105; A23L 33/40; A23L 33/19; A23L 33/185; A23L 33/175; A23L 33/125; A23L 33/115

(57) ABSTRACT

Methods of increasing microvascular blood flow in the muscle of a human subject comprise orally administering about 100 to about 800 mg cocoa flavanols per day in a nutritional composition comprising at least one source of protein, to a subject in need of increased microvascular blood flow in the muscle.

### FIELD

The present invention is directed to methods of increasing microvascular blood flow in muscle of a human subject by orally administering a nutritional composition comprising a high cocoa flavanol content. Aging adults, hospitalized or post-surgery patients, sarcopenic, diabetic, or malnourished subjects, or subjects suffering from a chronic gastrointestinal disorder, endothelial dysfunction, and/or vascular dysfunction may benefit from the inventive methods.

### BACKGROUND

Aging and chronic diseases such as cardiovascular diseases, metabolic syndrome, diabetes, and obesity, negatively impact the macrovascular blood flow (flow through larger arteries and arterioles) as well as microvascular flow (flow through capillary beds within peripheral tissues such as muscle). (see Mitchell, W. K., Phillips, B. E., Williams, J. P., Rankin, D., Smith, K., Lund, J. N., & Atherton, P. J. (2013). “Development of a new Sonovue™ contrast-enhanced ultrasound approach reveals temporal and age-related features of muscle microvascular responses to feeding.” Physiological Reports, 1 (5); Lind, L., and H. Lithell. 1993. “Decreased peripheral blood flow in the pathogenesis of the metabolic syndrome comprising hypertension, hyperlipidemia, and hyperinsulinemia.” Am. Heart J., 125:1494-1497; and Goodwill, A. G., and J. C. Frisbee. 2012. “Oxidant stress and skeletal muscle microvasculopathy in the metabolic syndrome.” Vascul. Pharmacol., 57:150-159).

The muscle microvasculature is the final interface through which circulating nutrients, oxygen, and hormones must pass from systemic circulation to the myocytes (muscle cells). Microvascular or capillary blood flow is also termed ‘nutritive flow’ since it is involved in transfer of nutrients to myocytes, in comparison to ‘non-nutritive’ flow or flow through vessels not in direct contact with myocytes. (see Clark, M. G., Wallis, M. G., Barrett, E. J., Vincent, M. A., Richards, S. M., Clerk, L. H., and Rattigan, S. 2003. “Blood flow and muscle metabolism: a focus on insulin action.” Am. J. Physiol. Endocrinol. Metab., 284 (2): E241-E258). At any given time, only about 30% of the capillaries in resting muscle are perfused, i.e., have blood flow. Microvascular flow is triggered in response to key signals such as a meal or exercise, with insulin being the major signaling molecule for endothelium-dependent vasodilation and relaxation of the terminal arterioles. Relaxation of the terminal arterioles results in “capillary recruitment” and an increase in distribution of blood within the tissue capillary bed. (see Clark, M. G., Wallis, M. G., Barrett, E. J., Vincent, M. A., Richards, S. M., Clerk, L. H., and Rattigan, S. 2003. “Blood flow and muscle metabolism: a focus on insulin action.” Am. J. Physiol. Endocrinol. Metab., 284 (2): E241-E258; Barrett, E. J., & Rattigan, S. (2012). “Muscle perfusion: its measurement and role in metabolic regulation.” Diabetes, 61 (11), 2661-2668). Blunted microvascular blood flow is thought to contribute to the age-related declines in muscle anabolic responses to feeding, also known as anabolic resistance. It has been suggested that anabolic resistance is caused by the reduced delivery of and/or utilization of insulin and amino acids in muscle, which eventually leads to loss of muscle mass, strength and function. (see Volpi, E., Mittendorfer, B., Rasmussen, B. B., & Wolfe, R. R. (2000). “The response of muscle protein anabolism to combined hyperaminoacidemia and glucose-induced hyperinsulinemia is impaired in the elderly.” The Journal of Clinical Endocrinology & Metabolism, 85 (12), 4481-4490; Clark, M. G., Wallis, M. G., Barrett, E. J., Vincent, M. A., Richards, S. M., Clerk, L. H., and Rattigan, S. 2003. “Blood flow and muscle metabolism: a focus on insulin action.” Am. J. Physiol. Endocrinol. Metab., 284 (2): E241-E258; and Timmerman, K. L., Dhanani, S., Glynn, E. L., Fry, C. S., Drummond, M. J., Jennings, K., et al. 2012. “A moderate acute increase in physical activity enhances nutritive flow and the muscle protein anabolic response to mixed nutrient intake in older adults.” Am. J. Clin. Nutr., 95 (6): 1403-1412).

Exercise, both in the form of acute isometric contractions and chronic resistance training, has been shown to increase both whole body blood flow as well as microvascular flow to muscle in older adults. (see Vincent, M. A., Clerk, L. H., Lindner, J. R., Price, W. J., Jahn, L. A., Leong-Poi, H., & Barrett, E. J. (2006). “Mixed meal and light exercise each recruit muscle capillaries in healthy humans.” American Journal of Physiology-Endocrinology And Metabolism, 290 (6): E1191-E1197; and Phillips, B. E., Atherton, P. J., Varadhan, K., Limb, M. C., Wilkinson, D. J., Sjøberg, K. A., Smith, K. & Williams, J. P. (2015). “The effects of resistance exercise training on macro-and micro-circulatory responses to feeding and skeletal muscle protein anabolism in older men.” The Journal of Physiology, 593 (12), 2721-2734). However, not all older adults have the ability and/or opportunity to exercise sufficiently, and especially those with mobility limitations may not be able to exercise to the extent necessary to reduce loss of muscle mass, strength and/or function.

Sodium nitroprusside, a nitric oxide donor, has been shown to increase microvascular flow and consequently muscle protein synthesis. (see Timmerman, K. L., Dhanani, S., Glynn, E. L., Fry, C. S., Drummond, M. J., Jennings, K., et al. 2012. “A moderate acute increase in physical activity enhances nutritive flow and the muscle protein anabolic response to mixed nutrient intake in older adults.” Am. J. Clin. Nutr., 95 (6): 1403-1412). However, sodium nitroprusside is indicated for use in very specialized treatment of high blood pressure, for example, in heart failure and during surgery, and is not suitable for chronic daily use in older adults or in many subjects otherwise experiencing reduced microvascular blood flow.

An intravenous infusion of 20 g free amino acids together with oral delivery of cocoa flavanols was shown to increase muscle blood volume (microvascular) as compared with an intravenous infusion of amino acids alone. (see Phillips, B. E., Atherton, P. J., Varadhan, K., Limb, M. C., Williams, J. P., & Smith, K. (2016), “Acute cocoa flavanol supplementation improves muscle macro-and microvascular but not anabolic responses to amino acids in older men.” Applied Physiology, Nutrition, and Metabolism, 41 (5), 548-556). However, intravenous infusion is not a common or convenient mode of food administration, except in a hospital or acute setting, and it is well known that intravenous administration and oral administration of food and drugs can result in different metabolic responses. (see Lickley, H. L. A., Track, N. S., Vranic, M., & Bury, K. D. (1978). “Metabolic responses to enteral and parenteral nutrition.” The American Journal of Surgery, 135 (2), 172-176).

Accordingly, convenient methods of increasing microvascular blood flow which can be implemented on a daily basis are desired.

### SUMMARY

The present invention overcomes one or more disadvantages of the prior art and provides improved methods for increasing microvascular blood flow.

In one embodiment, the invention is directed to methods of increasing microvascular blood flow in muscle of a human subject. The methods comprise orally administering about 100 to about 800 mg cocoa flavanols per day in a nutritional composition comprising at least one source of protein, to a subject in need of increased microvascular blood flow in muscle.

In additional embodiments, the methods are suitable for increasing microvascular blood flow in muscle of a human subject who consumes a low protein diet.

The methods of the invention are advantageous in providing a convenient method for increasing microvascular blood flow, which, in turn, can reduce loss of muscle mass, strength and/or function. The methods are advantageous in providing these benefits through oral, rather than intravenous, administration of a nutritional composition comprising protein, and not involving drug administration. Additionally, the methods are suitable for subjects with limited ability or opportunity for exercise, especially those with mobility limitations, and are useful for subjects who consume a low protein diet. The methods can be conducted on a daily basis as desired. These and additional advantages of the inventive methods will be more fully apparent in view of the detailed description.

### BRIEF DESCRIPTION OF THE DRAWING

Certain aspects of the invention are illustrated in the drawing, in which:

FIG. 1 shows results of contrast enhanced ultrasound (CEUS) measurement of muscle microvascular blood volume in experimental versus control subjects as described in the Example.

### DETAILED DESCRIPTION

While the general inventive concepts are susceptible of embodiment in many different forms, described herein in detail are specific embodiments of the invention, with the understanding that the present disclosure is to be considered as an exemplification of the principles of the general inventive concepts. Accordingly, the general inventive concepts are not intended to be limited to the specific embodiments illustrated and described herein.

In one embodiment, the invention is directed to methods of administering nutritional compositions. The term “nutritional composition” as used herein, unless otherwise specified, encompasses all forms of nutritional compositions, including nutritional liquids, including emulsified liquids, and liquids formed by reconstituting nutritional powders, for example, by addition of water, and nutritional solids, including, but not limited to those in powder form. The nutritional compositions are suitable for oral consumption by a human.

All percentages, parts and ratios as used herein, are by weight of the total composition, unless otherwise specified. All such weights as they pertain to listed ingredients are based on the active level and, therefore, do not include solvents or byproducts that may be included in commercially available materials, unless otherwise specified.

The terminology as set forth herein is for description of the embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

Throughout this specification, when a range of values is defined with respect to a particular characteristic of the present invention, the present invention relates to and explicitly incorporates every specific subrange therein. Additionally, throughout this specification, when a group of substances is defined with respect to a particular characteristic of the present invention, the present invention relates to and explicitly incorporates every specific subgroup therein. Any specified range or group is to be understood as a shorthand way of referring to every member of a range or group individually as well as every possible subrange or subgroup encompassed therein.

The various embodiments of the nutritional compositions employed in the methods of the present disclosure may also be substantially free of any optional or selected ingredient or feature described herein, provided that the remaining nutritional composition still contains all of the required ingredients or features as described herein. In this context, and unless otherwise specified, the term “substantially free” means that the selected nutritional product contains less than a functional amount of the optional ingredient, typically less than 1%, including less than 0.5%, including less than 0.1%, and also including zero percent, by weight, of such optional or selected essential ingredient.

The methods and nutritional compositions described herein may comprise, consist of, or consist essentially of the essential steps and elements, respectively, as described herein, as well as any additional or optional steps and elements, respectively, described herein. Any combination of method or process steps as used herein may be performed in any order, unless otherwise specified or clearly implied to the contrary by the context in which the referenced combination is made.

Unless otherwise indicated herein, all exemplary embodiments, sub-embodiments, specific embodiments and optional embodiments are respective exemplary embodiments, sub-embodiments, specific embodiments and optional embodiments to all embodiments described herein.

In one embodiment, the invention is directed to a method of increasing microvascular blood flow in muscle of a human subject. The subject is one in need of increased microvascular blood flow. For example, the subject may be an older adult, for example, over 40 years of age, over 50 years of age, over 60 years of age, over 65 years of age, over 70 years of age, or older. As discussed previously, older adults typically exhibit some reduction in microvascular blood flow and may encounter difficulties in preventing such a reduction by exercise alone. The Example presented herein demonstrates that the improvement of increased blood flow is achieved regardless of gender and is evident in both older men and older women. In additional embodiments, the subject may be experiencing an event that contributes to a reduction in microvascular blood flow, for example, hospitalization, surgery, immobility, or the like. In further embodiments, the subject may be sarcopenic, diabetic, or malnourished, or suffering from a chronic disease. For example, subjects with chronic gastrointestinal disorders, including cancer patients undergoing chemotherapy and encountering gastrointestinal disorders as a result, subjects with endothelial dysfunction, such as cardiovascular disease or other inflammatory disease states, and subjects with vascular dysfunction, for example, from a chronic disease such as diabetes, peripheral arterial disease (PAD), and peripheral vascular disease (PVD), are suitable subjects for improving microvascular blood flow to muscle according to the invention.

In a specific embodiment, the subject consumes a low protein diet. For example, the subject may have a daily protein intake of less than about 1.2 grams, less than about 1.0 gram, or less than about 0.8 grams of protein per kilogram of body weight. Consequently, the nutritional composition may comprise a relatively low amount of protein as described in further detail hereafter, yet still provide improved microvascular blood flow.

The inventive methods comprise orally administering about 100 to about 800 mg cocoa flavanols per day in a nutritional composition comprising at least one source of protein, to a subject in need of increased microvascular blood flow in muscle. Improved microvascular blood flow to muscle can reduce losses in muscle mass, strength and/or function. In more specific embodiments, the methods comprise orally administering about 150 to about 600 mg cocoa flavanols per day in the nutritional composition, orally administering about 200 to about 600 mg cocoa flavanols per day in the nutritional composition, or orally administering about 300 to about 600 mg cocoa flavanols per day in the nutritional composition.

The inventors have surprisingly discovered that oral administration of such dosages of cocoa flavanols with a small oral meal as provided by the nutritional composition comprising protein improves capillary recruitment in the muscle microvasculature, thereby improving microvascular blood flow to muscle. This is surprising as the intact protein from the nutritional composition must go through the processes of digestion and absorption before amino acids from the protein can reach muscle, and it could not be predicted or expected that orally administered protein, particularly in a low dose, and the cocoa flavanols would result in sufficient amino acids being delivered to the muscle to improve capillary recruitment in the muscle microvasculature, resulting in increased blood volume and thereby improving microvascular blood flow to muscle. As noted above, Phillips (2016) described a study in which an intravenous infusion of high doses of (20 g) free amino acids together with oral delivery of cocoa flavanols was shown to increase muscle blood volume beyond that caused by intravenous infusion of amino acids alone. Amino acids are needed to stimulate insulin response, and insulin signals the terminal arterioles to initiate capillary recruitment towards driving capillary blood flow. However, intravenous infusion of free amino acids as described by Phillips is quite different from, and not predictive of the response that would be obtained by oral administration of a nutritional composition containing intact protein since intravenous infusion of free amino acids bypasses the processes of protein digestion and absorption that are encountered when intact protein is delivered as part of a mixed oral meal.

In a specific embodiment, the indicated dosage of cocoa flavanol is provided by including high flavanol cocoa in the nutritional composition. Various high flavanol cocoa products are commercially available and suitable for use in the nutritional products employed in the inventive methods, including, but not limited to, high flavanol cocoa products from Mars Inc. and from Barry Callebaut. Such products may typically contain from about 20 to about 150 mg/g of epicatechin and from about 80 to about 600 mg/g of total flavanols. Regular cocoa, such as that employed for chocolate flavoring, on the other hand, typically includes about 1.2 mg/g of epicatechin and about 3.4 mg/g of total flavanols.

The indicated dosage of cocoa flavanols may be administered in a single serving or may be administered in multiple servings. In a specific embodiment, the indicated dosage of cocoa flavanols is administered in a single serving. The term “serving” as used herein, unless otherwise specified, refers to an amount which is intended to be consumed by an individual in one sitting or within one hour or less. While a typical nutritional composition serving may comprise 237 ml (8 ounces) of a liquid nutritional composition, the liquid nutritional compositions employed in the methods of the invention may be provided in smaller or larger servings as desired. For example, in one embodiment, a liquid nutritional composition serving may comprise from about 50 ml to about 300 ml, from about 50 ml to about 200 ml, or from about 50 ml to about 100 ml. In another embodiment, where the nutritional composition is a solid, for example, a solid powder, a nutritional composition serving may comprise from about 25 to about 100 g powder, or from about 25 to about 80 g powder. It should also be recognized that a serving of a liquid nutritional composition according to the invention may comprise a manufactured ready-to-drink liquid nutritional composition or a reconstituted liquid composition formed from a powder nutritional composition, for example, by addition of water.

In specific embodiments, the nutritional composition has a relatively low protein content, yet still provides improved microvascular blood flow. For example, in specific embodiments, the source of protein comprises from about 1 wt % to about 25 wt % of the nutritional composition. In more specific embodiments, the source of protein comprises from about 2 wt % to about 25 wt % of the nutritional composition, including about 2 wt % to about 20 wt %, about 2 wt % to about 15 wt %, about 5 wt % to about 20 wt %, about 5 wt % to about 25 wt %, about 10 wt % to about 25 wt %, or about 5 wt % to about 15 wt % of the nutritional composition.

In additional specific embodiments, the nutritional composition is a liquid and comprises protein in an amount of from about 2 to about 16 g, from about 2 to about 10 g, or from about 2 to about 8 g, per 100 ml of the liquid nutritional composition. In other embodiments, the nutritional composition is a powder and comprises protein in an amount of from about 3 to about 20 g, or from about 5 to about 20 g, per 100 g of the powder nutritional composition.

One or more sources of protein may be included in the nutritional composition. A wide variety of sources and types of protein can be used in the nutritional compositions which are employed in the methods of the invention. For example, the source of protein may include, but is not limited to, intact, hydrolyzed, and partially hydrolyzed protein, which may be derived from any suitable source such as milk (e.g., casein, whey), animal (e.g., meat, fish), cereal (e.g., rice, brown rice, corn, barley, etc.), vegetable (e.g., soy, pea, yellow pea, fava bean, chickpea, canola, potato, mung, ancient grains such as quinoa, amaranth, and chia, hamp, flax seed, etc.), and combinations of two or more thereof. The protein may also include one or a mixture of amino acids (often described as free amino acids) known for use in nutritional products, and/or metabolites thereof, or a combination of one or more such amino acids and/or metabolites, with the intact, hydrolyzed, and partially hydrolyzed proteins described herein. The amino acids may be naturally occurring or synthetic amino acids.

More specific examples of sources of protein which are suitable for use in the exemplary nutritional compositions described herein include, but are not limited to, whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins such as beef protein isolate and/or chicken protein isolate, potato proteins, chickpea proteins, canola proteins, mung proteins, quinoa proteins, amaranth proteins, chia proteins, hamp proteins, flax seed proteins, earthworm proteins, insect proteins, and combinations of two or more thereof. Suitable amino acids may be naturally occurring or synthetic amino acids. In one embodiment, one or more branched chain amino acids (leucine, isoleucine and/or valine) and/or one or more metabolites of branched chain amino acids, for example, leucic acid (also known as a-hydroxyisocaproic acid or HICA), keto isocaproate (KIC), and/or β-hydroxy-β-methylbutyrate (HMB), are included as a protein in the nutritional compositions. The nutritional compositions can include any individual source of protein or combination of any of the various sources of protein listed above.

The nutritional compositions may also comprise carbohydrate and/or fat. In one embodiment, the nutritional compositions used in the methods of the invention comprise both carbohydrate and fat.

In specific embodiments, a source of carbohydrate is present in an amount from about 5 wt % to about 75 wt % of the nutritional composition. In more specific embodiments, the source of carbohydrate is present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In a specific embodiment, wherein the nutritional composition is a liquid, the source of carbohydrate comprises about 5 wt % to about 30 wt % of the nutritional composition. In more specific liquid embodiments, the carbohydrate comprises about 5 wt % to about 25 wt %, about 5 wt % to about 20 wt %, about 5 wt % to about 15 wt %, about 10 wt % to about 25 wt %, about 10 wt % to about 20 wt %, about 15 wt % to about 25 wt %, or about 15 wt % to about 30 wt % of the nutritional composition. In another specific embodiment, wherein the nutritional composition is a powder, the source of carbohydrate comprises about 25 wt % to about 75 wt % of the nutritional composition. In more specific powder embodiments, the carbohydrate comprises about 30 wt % to about 70 wt %, about 35 wt % to about 65 wt %, about 40 wt % to about 65 wt %, about 40 wt % to about 70 wt %, about 50 wt % to about 70 wt %, or about 50 wt % to about 75 wt % of the nutritional composition.

Sources of carbohydrates suitable for use in the nutritional compositions may be simple or complex, or variations, or combinations thereof. Various sources of carbohydrate may be used so long as the source is suitable for use in a nutritional composition and is otherwise compatible with any other selected ingredients or features present in the nutritional composition. Non-limiting examples of sources of carbohydrates suitable for use in the nutritional compositions include maltodextrin, hydrolyzed or modified starch, hydrolyzed or modified cornstarch, glucose polymers such as polydextrose and dextrins, corn syrup, corn syrup solids, rice-derived carbohydrates such as rice maltodextrin, brown rice mild powder and brown rice syrup, sucrose, glucose, fructose, lactose, high fructose corn syrup, honey, sugar alcohols (e.g., maltitol, erythritol, sorbitol), isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, Fibersol™, fruit puree, vegetable puree, isomalto-oligosaccharides, monosaccharides, disaccharides, tapioca-derived carbohydrates, inulin, other digestion-resistant starches, and artificial sweeteners, and combinations of two or more thereof. The nutritional compositions may include any individual source of carbohydrate or combination of any of the various sources of carbohydrate listed above.

The term “fat” as used herein, unless otherwise specified, refers to lipids, fats, oils, and combinations thereof. In specific embodiments, the nutritional composition comprises about 0.5 wt % to about 20 wt % of a source of fat. In more specific embodiments, the source of fat comprises about 0.5 wt % to about 18 wt % of the nutritional composition, including about 0.5 wt % to about 15 wt %, about 0.5 wt % to about 10 wt %, about 0.5 wt % to about 5 wt %, about 2 wt % to about 8 wt %, about 2 wt % to about 10 wt %, about 5 wt % to about 15 wt %, or about 5 wt % to about 20 wt % of the nutritional composition.

Sources of fat suitable for use in the nutritional composition include, but are not limited to, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, lecithin, and long chain polyunsaturated fatty acids such as docosahexanoic acid (DHA), arachidonic acid (ARA), docosapentaenoic acid (DPA), eicosapentaenoic acid (EPA), and combinations thereof. The nutritional compositions can include any individual source of fat or combination of any of the various sources of fat listed above.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may further comprise one or more additional components that may modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

Additionally, the nutritional composition may further include vitamins or related nutrients, non-limiting examples of which include vitamin A, vitamin B12, vitamin C, vitamin D, vitamin K, thiamine, riboflavin, pyridoxine, niacin, folic acid, pantothenic acid, biotin, choline, inositol, salts and derivatives thereof, and combinations thereof. Water soluble vitamins may be added in the form of a water-soluble vitamin (WSV) premix and/or oil-soluble vitamins may be added in one or more oil carriers as desired.

In additional embodiments, the nutritional composition may further include one or more minerals, non-limiting examples of which include calcium, phosphorus, magnesium, zinc, manganese, sodium, potassium, molybdenum, chromium, chloride, and combinations thereof.

In additional embodiments, the nutritional composition may further include one or more probiotics. The term “probiotic” as used herein refers to a microorganism such as a bacteria or yeast that survives the digestive process to confer a health benefit to the subject. Examples of probiotics that can be included in the nutritional compositions, either alone or in combination, include, but are not limited to, Bifidobacterium (B.), such as B. breve, B. infantis, B. lactis, B. bifidum, B. longum, and B. animalis, and Lactobacillus (L.), such as L. rhamnosus, L. acidophilus, L. fermentum, L. reuteri, Streptococcus thermophilus, Akkermansia, Bacteroides, Enterococcus, Eubacterium, Fecalibacterium, Roseburia, and/or Saccharomyces.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The high flavanol cocoa component may be added at any time as desired in the process, for example, to the aqueous solution or to the emulsified blend. The composition may be spray-dried or otherwise dried, if a powder product is desirable. Alternatively, a powder product can be formed by dry blending ingredients, in which case the high flavanol cocoa component may be dry blended with one or more dry ingredients.

The following Example demonstrates aspects of the inventive methods.

### EXAMPLE

This example illustrates a specific embodiment of the method of the invention, is provided solely for the purpose of illustration, and is not to be construed as limiting of the general inventive concepts, as many variations thereof are possible without departing from the spirit and scope of the general inventive concepts.

A clinical study was conducted using an orally administered low-protein nutritional composition. Study subjects included both men and women at least 65 years of age. For both a Control group (n=12) and an Experimental group (n=12), subjects were administered 100 ml of a liquid nutritional composition delivering a low dose, about 6 g, of protein (milk protein isolate, calcium and sodium caseinates, and soy protein isolate), delivering about 2.66 g essential amino acids), about 20 g carbohydrates (corn maltodextrin, corn syrup, sucrose, cellulose stabilizer and carboxymethyl cellulose stabilizer, containing about 6.5 g sugar) and about 5 g fat (canola oil, corn oil and lecithin). In the Experimental group, the subjects were also provided with about 33 g of chocolate chips composed of high flavanol cocoa extract (Acticoa-Barry Callebaut) delivering 500 mg total cocoa flavonoids 30 minutes prior to administration of the liquid nutritional composition.

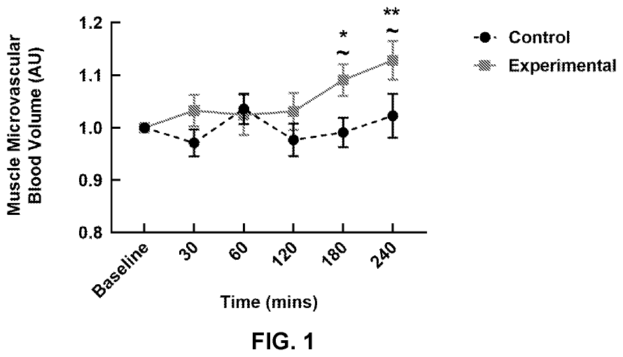

Muscle blood flow was measured in the vastus lateralis using contrast enhanced ultrasound (CEUS), at baseline (prior to introduction of the nutritional intervention), and at 30 min, 1 hr, 2 hr, 3 hr and 4 hr post-feeding. The CEUS technique measured intra-muscle blood volume (A− value), which is the blood volume inside the capillaries bed of the region of interest within the large leg muscle (vastus lateralis), i.e., the smallest blood vessels that lie deep inside the muscle tissue bed. The results are set forth in FIG. 1, presented as Mean±SEM

FIG. 1 shows the normalized muscle blood volume (MBV) changes from baseline (BL) in the vastus lateralis in response to treatments. Only the Experimental group demonstrated a significant increase from baseline in MBV responses to the meal, with this increase evident at 180 and 240-min post-meal (BL: 1.0 (normalized) vs. 180-min: 1.09±0.03, p=0.0462, and vs. 240-min: 1.13±0.04, p=0.0023, 2-way repeated measure ANOVA with Dunnett's post-hoc analysis). For Control, there was no significant change from baseline over time (BL: 1.0 (normalized) vs. 180-min: 0.99±0.03, non-significant (NS), and vs. 240-min: 1.02±0.04, NS). AU=Arbitrary units.

MBV was significantly higher in the Experimental group versus Control group post-feeding at 180 min (Cocoa: 1.09±0.03 vs. Control: 0.99±0.03, p<0.0329) and 240-min (Cocoa: 1.13±0.04 vs. Control: 1.02±0.04, p=0.0206, 2-way repeated measure ANOVA with Sidak's post-hoc analysis).

These data are surprising in that they demonstrate that oral delivery of cocoa flavanols with a low protein meal can cause an increase in blood flow into the muscle capillary bed that is needed for nutrient transport to the myocytes (muscle cells). This increase in muscle blood flow into the capillary bed is mediated by capillary recruitment leading to expanded volume of blood through the muscle tissue bed. The increased microvascular blood flow can lead to preservation of muscle mass, strength and/or function in a subject.

Thus the inventive methods are advantageous in providing a convenient method for improving skeletal muscle microvascular blood flow.

While the present application has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and processes, or illustrative examples shown and described. Accordingly, departures may be made from such details without departing from the spirit or scope of the general inventive concept.

1. A method of increasing microvascular blood flow in muscle of a human subject, comprising orally administering about 100 to about 800 mg cocoa flavanols per day in a nutritional composition comprising at least one source of protein, to a subject in need of increased microvascular blood flow in muscle.

2. The method of claim 1, comprising orally administering about 200 to about 600 mg cocoa flavanols per day in the nutritional composition.

3. The method of claim 1, wherein the subject is an adult over 40 years of age.

4. The method of claim 1, wherein the subject is a hospitalized patient, a post-surgery patient, sarcopenic, diabetic, malnourished, and/or suffering from a chronic gastrointestinal disorder, endothelial dysfunction, and/or vascular dysfunction.

5. The method of claim 1, wherein the subject has a daily protein intake of less than about 1.2 grams, less than about 1.0 gram, or less than about 0.8 gram, of protein per kilogram of body weight.

6. The method of claim 1, wherein the nutritional composition comprises protein, fat and carbohydrate.

7. The method of claim 1, wherein the nutritional composition is a liquid and comprises protein in an amount of from about 2 to about 16 g, or from about 2 to about 10 g, per 100 ml of the liquid nutritional composition.

8. The method of claim 7, wherein the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition.

9. The method of claim 1, wherein the nutritional composition is a powder and comprises protein in an amount of from about 3 to about 20 g, or from about 5 to about 20 g, per 100 g of the powder nutritional composition.

10. The method of claim 9, wherein the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition

11. The method of claim 1, wherein the source of protein comprises one or more amino acids and/or one or more metabolites of amino acids, more specifically, one or more branched chain amino acids and/or one or more metabolites of branched chain amino acids, or, more specifically, one or more of leucine, isoleucine, valine, leucic acid, keto isocaproate or β-hydroxy-β-methylbutyrate.

12. The method of claim 1, wherein the source of protein comprises at least one of milk protein, animal protein, cereal protein, or vegetable protein, or a combination of two or more thereof.

13. The method of claim 1, wherein the source of protein comprises at least one selected from whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm proteins, insect proteins, or combinations of two of more thereof.

14. The method of claim 1, wherein the nutritional composition comprises at least one fat source selected from algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, lecithin, long chain polyunsaturated fatty acids, and combinations of two of more thereof.

15. The method of claim 1, wherein the nutritional composition comprises at least one carbohydrate source selected from maltodextrin, hydrolyzed starch, modified starch, hydrolyzed cornstarch, modified cornstarch, polydextrose, dextrins, corn syrup, corn syrup solids, rice maltodextrin, brown rice mild powder, brown rice syrup, sucrose, glucose, fructose, lactose, high fructose corn syrup, honey, maltitol, erythritol, sorbitol, isomaltulose, sucromalt, pullulan, potato starch, corn starch, fructooligosaccharides, galactooligosaccharides, human milk oligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, fiber, fruit puree, vegetable puree, isomalto-oligosaccharides, monosaccharides, disaccharides, tapioca-derived carbohydrates, inulin, and artificial sweeteners, and combinations of two of more thereof.

16. The method of claim 1, wherein the nutritional composition comprises one or more probiotics.
